(19) United States
(12) Patent Application Publication
(10) Pub. No.: US 2022/0071618 A1
(43) Pub. Date: Mar. 10, 2022

(54) SURGICAL NEEDLE GUIDING ROD AND KIT

(71) Applicant: José António PACHECO DOS SANTOS DIAS, Lisboa (PT)

(72) Inventor: José António PACHECO DOS SANTOS DIAS, Lisboa (PT)

(21) Appl. No.: 17/418,869

(22) PCT Filed: Dec. 17, 2019
(86) PCT No.: PCT/IB2019/060932

§ 371 Date: Jun. 28, 2021

(30) Foreign Application Priority Data
Dec. 27, 2018 (PT) 115233

(51) Int. Cl. A61B 17/04; A61B 17/11; A61B 17/062

(52) U.S. Cl. CPC A61B 17/0482; A61B 17/062; A61B 17/11

(57) ABSTRACT

The present invention in the area of medical devices, for prostate and urethral surgery, namely providing anastomosis between the bladder and the urethra of a patient. It is an object of the invention a surgical needle guiding rod (1), which his solid and comprises a proximal portion (2) and a distal portion (3), a first opening (11) and a second opening (12) being formed in the surface of the distal portion (3) thereby forming a channel (4) through the solid rod (1) between the two openings, the channel (4) being such that it allows a surgical needle to pass by entering the one of the two openings and exiting the other opening. The rod (1) of the present invention thus is solid, only requiring the channel (4) between the two openings formed in the distal portion (3) to provide for suturing of urethral-bladder tissues and thereby facilitating the anastomosis procedure.

### FIELD OF THE INVENTION

The present invention is enclosed in the area of medical devices, in particular for prostate and urethral surgery, namely for providing anastomosis between the bladder and the urethra of a patient.

### PRIOR ART

Prostate cancer is the most common cancer in men in developed countries. It is the second leading cause of cancer death in men in these countries.

Presently, one of the most frequently performed treatments for the treatment of this type of cancer is surgery, called radical prostatectomy. It can be done openly, laparoscopically or robotically. The tendency to perform this surgery through one of these last two approaches (laparoscopic and robotic) is increasing, given the lower aggressiveness, less invasiveness, lower morbidity and faster recovery of patients.

However, when performed by the latter methodologies and despite the significant advantages to the patient, surgery is technically more demanding for surgeons. One of the technically most difficult steps of prostate cancer surgery is the anastomosis (junction, union) of the bladder with the urethra (called vesico-urethral anastomosis).

It is a very delicate and difficult step, which is performed at the end of the intervention—which means that sometimes it is done after 2 or 3 hours of surgery —, when the surgeons are already more tired, increasing the difficulty of accomplishing some of the gestures needed to complete this step.

Devices known in the art are unsuitable for such purpose. In particular, it is the case of accessories designed for other purposes which are used by urologists to somehow attempt to make this step easier: they are metallic accessories, similar to urethral dilators, with a circular end opening.

This opening is used to facilitate insertion of the tip of the needle into the urethral lumen. However, it does not allow the gesture of insertion of the needle tip through the urethra and consequent rotation of the needle (necessary for the correct and adequate accomplishment of this step) to be done swiftly, safely and effectively, guaranteeing to involve the necessary and sufficient extension of the urethra to ensure a union to the bladder with the necessary resistance to the subsequent function of these organs, often causing a union between the urethra and the bladder that does not become tight, which tears the very delicate urethral tissue and sometimes contributes to aggravate the problem of incontinence that often arises after this type of surgery.

It is the case of the object described in U.S. Pat. No. 2,897,820.

Thus, the present invention provides a solution to the described issues found in prior art.

### SUMMARY OF THE INVENTION

It is an object of the present invention a surgical needle guiding rod (1), such rod (1) being solid and comprising a proximal portion (2) and a distal portion (3), a first opening (11) and a second opening (12) being formed on the surface of the distal portion (3) thereby forming a channel (4) through the solid rod (1) between the two openings, the channel (4) being such that it allows a surgical needle to pass by entering the one of the two openings and exiting the other opening. By having a channel (4) formed between the two openings, in the distal portion (3), the rod (1) of the present invention provides for suturing of urethral-bladder tissues and thereby facilitates the anastomosis procedure. Thus, a surgeon may position the rod (1) of the present invention as desired and the configuration of openings allows that a surgical needle inserted in the first opening (11) will be directed into the second opening (12), which would be positioned so to be adjacent to the tissue which the surgeon wishes to suture.

Advantageously, it has a curved cross-section, preferably circular, the first opening (11) being formed in the tip of the rod (9) an in a substantially concentric fashion with the axis of the rod (1), and the second opening (12) being formed laterally on the surface of the rod (1). Thus, a curved needle may easily be inserted in the first opening (11), provided in the tip of the rod (9), and exit the rod (1) in the second opening (12), provided on the side. Thus, the device of the present invention—resorting to a simple construction—provides for the guiding of a surgical needle from the entrance of the urethra [where the first opening (11) will be positioned] to its lateral tissue [where the second opening (12) is].

In yet another inventive aspect of the rod (1) of the present invention, the first opening (11) is wider than the second opening (12), the channel (4) forming a substantially conical shape between the two openings. Therefore, the first opening (11), being wider, provides an easiness of insertion of the needle, and the corresponding shape of the channel (4) (substantially conical) and the second opening (12) (lesser width), allows for the needle to be directed into the second opening (12), to the correct position of the urethral tissue, thus enhancing the anastomosis operation.

In yet another inventive aspect of the rod (1) of the present invention, it further comprises adhering/fixation means (6) provided in the distal portion (3) and actuation means in the proximal portion (2), the adhering/fixation means (6) being such that, when applied in the urethra of a patient and under an actuation action in the actuation means, provide adherence to the urethra of a patient allowing the rod (1) to push and fix the urethra and perineum. Such adhering and actuation means may take several different forms, all providing for i) pushing of the perineum and urethra into a desired position and ii) maintaining a correct position of the surgical rod (1) in an anastomosis operation, as defined by a surgeon. After positioning and fixation by operation of the actuation means, suturing as previously described may be performed (additionally) much more easily and without the risk that changes occur in the positioning of the urethra with respect to the distal portion (3) of the rod (1).

It is also an object of the present invention a surgical suture kit comprising the rod (1) of any of the preceding claims and a surgical needle, such surgical needle having a curvature, the curvature preferably being of ⅜ circle, ½ circle or ⅝ circle, more preferably with a chord length of 16 to 30 mm.

### DESCRIPTION OF FIGURES

FIG. 1—frontal view of a representation of an embodiment of the guiding rod (1) of the present invention, specifically of a part of the distal portion (3), in which the second opening (12) and the slot (5) are viewable. The channel (4) between the second opening (12) and the first opening (11) is represented in streak. In such case, the first opening (11) is wider than the second opening (12), providing a conical channel (4), and the slot (5) allows for an easier removal of a needle.

FIG. 2—section view of a representation of an embodiment of the guiding rod (1) of the present invention, specifically of a part of the distal portion (3), in which the second opening (12), the first opening (11) and the channel (4) are viewable. The channel (4) has a conical and curved shape.

FIG. 3—side view of a representation of an embodiment of the guiding rod (1) of the present invention, containing both the proximal portion (2) and the distal portion (3), such rod (1) having a distal portion (3) according to the embodiments of FIGS. 1 and 2. The rod (1) further comprises a proximal button (7) which activates the rotation of the distal tip section (8).

FIGS. 4a and 4b—representation of the actuation of the embodiment of FIG. 3, providing the rotation of the distal tip section (8).

FIG. 5—representation of a second actuation of the embodiment of FIG. 3, providing wherein actuation means, which in such case consist of the proximal button (7), provide inflation of a balloon or protrusion from the surface of bulking parts [the adhering/fixation means (6)].

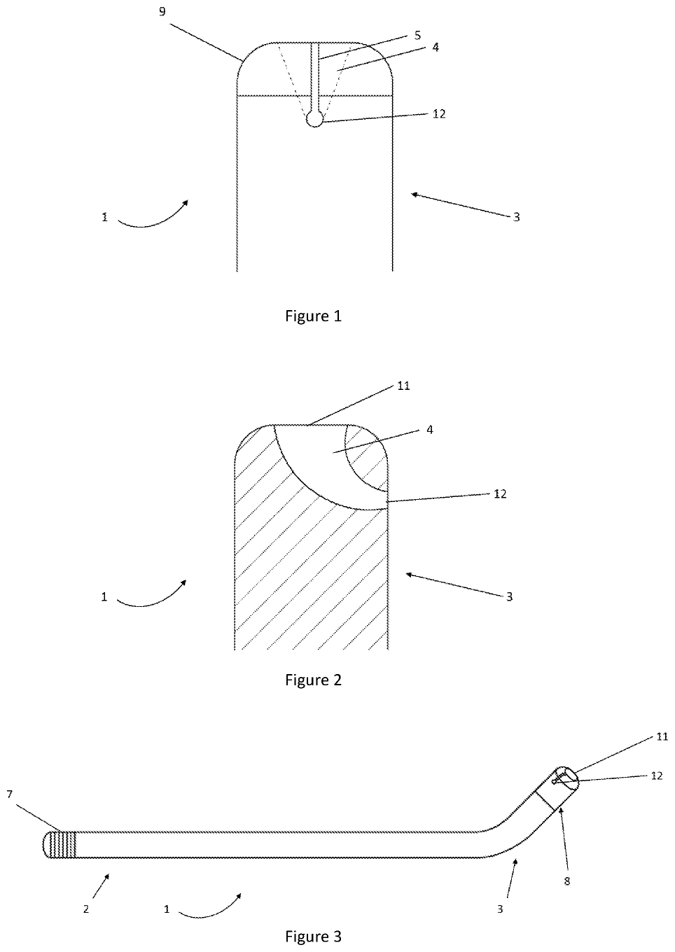

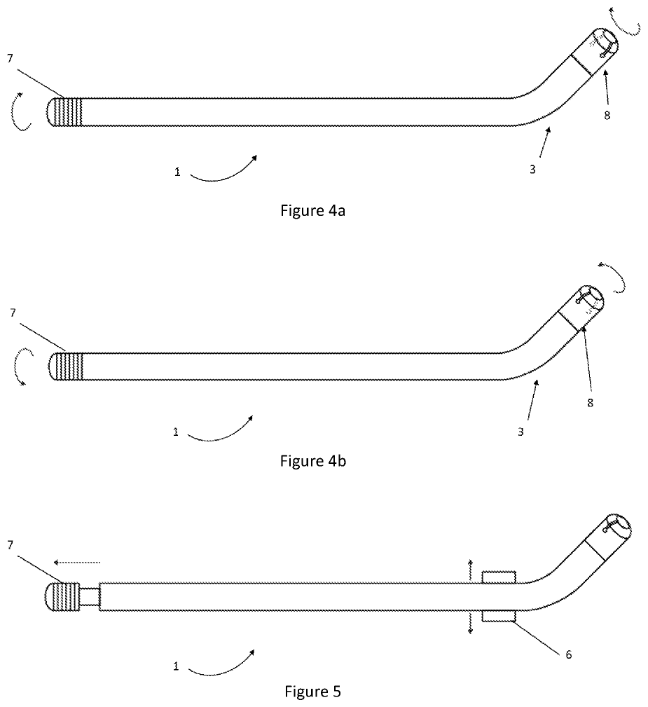

### DETAILED DESCRIPTION

The more general and advantageous configurations of the present invention are described in the Summary of the invention. Such configurations are detailed below in accordance with other advantageous and/or preferred aspects of the present invention.

In preferred embodiment of the rod (1) of the present invention, the channel (4) is curved. The curvature provides an enhanced guidance of a surgical needle. An implementation of such embodiment is shown in FIG. 2.

In another preferred embodiment of the rod (1) of the present invention, combinable with any above described, it has a straight section and a curved section, the curved portion comprising the distal section and the straight portion comprising the proximal section. Such curved section provides for easiness of insertion of the device, as well as it enhances the positioning of the first (11) and second openings (12), for subsequent suturing.

In another inventive aspect of the rod (1) of the present invention, a slot (5) is formed on the surface of the rod (1) between the two openings and the channel (4), the width of the slot (5) being less than the width of the second opening (12). A sowing string is connected to the needle that passes through the channel (4), and the slot (5) thereby allows for an eased removal of the needle.

Preferably, the width of the first opening (11) is of 3-6 mm, and the width of the second opening (12) is of 2-4 mm. Preferably, the width of its cross section is of 7-9 mm, preferably 8 mm.

In another inventive aspect of the rod (1) of the present invention, it further comprises a proximal button (7) provided in the tip of the proximal portion (2) and a transmission mechanism, and a distal tip section (8) comprising the two openings is rotatable, where the proximal button (7) and the transmission mechanism are so arranged that, under rotation of the proximal button (7), the transmission mechanism provides rotation of the distal tip section (8) and, consequently, of the two openings and the respective channel (4) formed therein. Such embodiment provides for an easiness of operation, which does not require the surgeon to rotate the whole rod (1) to pass from a position of the second opening (12)—from which the needle exits the channel (4)—to a second position, to continue the suturing operation. With such transmission mechanism and proximal button (7), the distal tip section (8)—a section defined by comprising the two openings (first and second)—also rotates and therefore allows to position the second opening (12) adjacently to another section of the urethra.

In an embodiment of the rod (1) of the present invention, it is made of a metallic material suitable for human surgical application, preferably stainless steel or titanium. Such material is thus reusable.

In another—alternative—embodiment of the rod (1) of the present invention, it is made of a polymer material suitable for human surgical application, preferably polyether block amide, polytetramethylene glycol, an elastomer (a thermoplastic elastomer such as thermoplastic polyurethane, polyester elastomers, silicones, or polyethylene). Such material is thus not reusable.

In another embodiment of the rod (1) of the present invention, the proximal portion (2) comprises gripping means, preferably provided in the tip of the proximal portion (2).

Reference is made to the embodiment of the rod (1) which further comprises adhering/fixation means (6) provided in the distal portion (3) and actuation means in the proximal portion (2), the adhering/fixation means (6) being such that, when applied in the urethra of a patient and under an actuation action in the actuation means, provide adherence to the urethra of a patient. In a specific embodiment, the adhering/fixation means (6) consist of a balloon or a ring, arranged over the surface of the rod (1) and the actuation means of an element configured to provide inflation of such balloon, whereby an inflation channel (4) is formed inside the rod (1) between such inflation element and the inner side of the balloon.

Preferably, said inflation element consists of i) an inflation opening in the tip of the proximal portion (2) and connected to the inflation channel (4) and ii) an inflation element, preferably a syringe, connectable to said inflation opening whereby, under actuation of the inflation element while connected to the inflation opening, the balloon fills with a saline solution.

In another—alternative—specific embodiment, the adhering/fixation means (6) consist of at least three hinged arms or bulking parts which in an unarmed position are arranged at the surface of the rod (1) and, in an armed position reached under said actuation, protrude from the surface, the actuation means consisting of an element configured to provide actuation of said hinged arms.

Preferably, said element configured to provide actuation of said hinged arms consists of a button and a transmission mechanism connected between the button and the hinged arms and displaced on the inside of the rod (1) whereby, under actuation of the button, the transmission mechanism provides said protrusion of the hinged arms from the surface.

As will be clear to one skilled in the art, the present invention should not be limited to the embodiments described herein, and a number of changes are possible which remain within the scope of the present invention.

Of course, the preferred embodiments shown above are combinable, in the different possible forms, being herein avoided the repetition all such combinations.

1. A surgical needle guiding rod (1) characterised in that it is solid and comprises a proximal portion (2) and a distal portion (3), a first opening (11) and a second opening (12) being formed on the surface of the distal portion (3) thereby forming a channel (4) through the solid rod (1) between the two openings, the channel (4) being such that it allows a surgical needle to pass by entering one of the two openings and exiting the other opening.

2. A rod (1) according to the previous claim wherein it has a curved cross-section, preferably circular, the first opening (11) being formed in the tip of the rod (9) an in a substantially concentric fashion with the axis of the rod (1), and the second opening (12) being formed laterally on the surface of the rod (1).

3. A rod (1) according to the previous claim wherein the first opening (11) is wider than the second opening (12), the channel (4) forming a substantially conical shape between the two openings.

4. A rod (1) according to claim 1 wherein the channel (4) is curved.

5. A rod (1) according to claim 1 wherein it has a straight section and a curved section, the curved portion comprising the distal section and the straight portion comprising the proximal section.

6. A rod (1) according to claim 1 wherein a slot (5) is formed on the surface of the rod (1) between the two openings and the channel (4), the width of the slot (5) being less than the width of the second opening (12).

7. A rod (1) according to claim 1 wherein the width of the first opening (11) is of 1-4 mm, and the width of the second opening (12) is of 2-8 mm.

8. A rod (1) according to claim 1 wherein the width of its cross section is of 4-9 mm, preferably 8 mm.

9. A rod (1) according to claim 1 wherein it further comprises adhering/fixation means (6) provided in the distal portion (3) and actuation means in the proximal portion (2), the adhering/fixation means (6) being such that, when applied in the urethra of a patient and under an actuation action in the actuation means, provide adherence to the urethra of a patient thereby allowing the rod (1) to push and fix the urethra and perineum wherein, preferably:
the adhering/fixation means (6) consist of a balloon arranged over the surface of the rod (1) and the actuation means of an element configured to provide inflation of such balloon, whereby an inflation channel (4) is formed inside the rod (1) between such inflation element and the inner side of the balloon, and, more preferably:
said inflation element consists of i) an inflation opening in the tip of the proximal portion (2) and connected to the inflation channel (4) and ii) an inflation element, preferably a syringe, connectable to said inflation opening whereby, under actuation of the inflation element while connected to the inflation opening, the balloon fills with a saline solution.

10. A rod (1) according to claim 1 wherein it further comprises adhering/fixation means (6) provided in the distal portion (3) and actuation means in the proximal portion (2), the adhering/fixation means (6) being such that, when applied in the urethra of a patient and under an actuation action in the actuation means, provide adherence to the urethra of a patient wherein, preferably:
the adhering/fixation means (6) consist of at least three hinged arms or bulking parts which in an unarmed position are arranged at the surface of the rod (1) and, in an armed position, reached under said actuation, protrude from the surface, and the actuation means consisting of an element configured to provide actuation of said hinged arms and, more preferably:
said element configured to provide actuation of said hinged arms or bulking parts consists of a button and a transmission mechanism connected between the button and the hinged arms or bulking parts and displaced on the inside of the rod (1) whereby, under actuation of the button, the transmission mechanism provides said protrusion of the hinged arms from the surface.

11. A rod (1) according to claim 1 wherein it further comprises a proximal button (7) provided in the tip of the proximal portion (2) and a transmission mechanism, and a distal tip section (8) comprising the two openings is rotatable, where the proximal button (7) and the transmission mechanism are so arranged that, under rotation of the proximal button (7), the transmission mechanism provides rotation of the distal tip section (8) and, consequently, of the two openings and the respective channel (4) formed therein.

12. A rod (1) according to claim 1 wherein it is made of a metallic material suitable for human surgical application, preferably stainless steel or titanium.

13. A rod (1) according to claim 1 wherein it is made of a polymeric material, preferably polyether block amide, polytetramethylene glycol, an elastomer, or polyethylene.

14. A rod (1) according to claim 1 wherein the proximal portion (2) comprises gripping means, preferably provided in the tip of the proximal portion (2).

15. A surgical suture kit comprising the rod (1) of claim 1 and a surgical needle, such surgical needle having a curvature.
